(19) United States
(12) Patent Application Publication
(10) Pub. No.: US 2010/0136525 A1
(43) Pub. Date: Jun. 3, 2010

(54) FLUID ANALYSIS DEVICE AND METHOD

(75) Inventors: Jacobus Frederik Molenaar, Utrecht (NL); Adrianus Wilhelmus Dionisius Maria Van Den Bijgaart, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/993,508

(22) PCT Filed: Jun. 23, 2006
(86) PCT No.: PCT/IB2006/052060

§ 371 Date: Dec. 21, 2007

(30) Foreign Application Priority Data
Jun. 30, 2005 (EP) 05105884.0

(51) Int. Cl. C12Q 1/68; C12M 1/34; C12Q 1/02

(52) U.S. Cl. 435/6; 435/287.1; 435/287.2; 435/29

(57) ABSTRACT

The invention provides an analysis device (1) and method, that enables repeatedly pumping a sample fluid (50) with one or more analytes through a substrate (10) with one or more binding materials, even when the wetted substrate has a relatively low bubble pressure. Thereto, the device (1) comprises a first volume (16) and a second volume (18), and a return channel (22) with a valve (24) as well as a pump (20). The pump (20) can establish a pressure difference between the first volume (16) and the second volume (18), to pump the sample fluid (50) through the substrate (10), thereby allowing analyte to bind to the substrate. By opening the valve (24) and establishing a reversed pressure difference, sample fluid (50) bypasses the substrate (10) through the return channel (22). After closing the valve, the device is ready for a subsequent cycle, if desired. The device and method allow the analysis of in principle any amount of sample fluid, and the use of any type of substrate with respect to bubble pressure, and is thus more versatile and robust.

The present invention generally relates to fluid handling, in particular in a device or method for analysis.

Before describing the invention in more detail, first some background information is provided, that may be helpful in further understanding the invention.

Analysis is often performed in order to investigate qualitatively and/or quantitatively the composition of a (liquid) sample, in particular relating to the detection of the presence, absence or amount of specific DNA or RNA sequences or proteins in a sample. Especially PCR, the Polymerase Chain Reaction has contributed enormously to the development of assays of all types for the detection of the presence or absence of DNA or RNA sequences. At present, it is possible to collect DNA containing samples from an organism and determine the presence, absence or amount therein of specific DNA sequences. Technology is available to perform such analysis for multiple target sequences at the same time, so-called multiplex detection of target sequences to thereby increase throughput.

For example, for detection of specific bacteria in a blood sample or the like, a detection method is known which is based on a DNA multiplication process and binding of this DNA to fluorescent tracer molecules. Only specific types of DNA will bind to specific probe molecules. The presence of bound DNA is then detected by optical means, e.g. activation by a light source and detection by a camera.

At present, this type of analysis is not yet performed on a routine basis, such as for instance the measurement of the blood-glucose content in the case of diabetes. Generally, well-equipped laboratories are necessary, and careful protocols have to be used in order to avoid cross-contamination and to ensure that the results obtained are reliable i.e. false-positive or false-negative readings of the tests are minimized. Still a lot of manual labour is involved of extensively trained and supervised personnel.

The detection of the presence, absence or amount of DNA and/or RNA is indicative, for instance, for the presence, absence or amount of a gene, an allele of a gene, a genetic trait or disorder, a polymorphism, a single nucleotide polymorphism (SNP) or of the presence of exogenous DNA or RNA in an organism, i.e. the presence, absence or amount of pathogens or bacteria in organisms.

In particular, in a first aspect, the present invention relates to an analysis device for analyzing a sample fluid for the presence or amount of an analyte in the sample, the analysis device comprising a substrate having a first surface and an opposite second surface, and having a plurality of through going channels from the first surface to the second surface, and at least partially being provided with a binding substance specific for the analyte, a first volume in fluid communication with the first surface, a second volume in fluid communication with the second surface, and a pump for establishing a pressure difference between the first volume and the second volume, the pump being operatively connected to the first volume.

Document U.S. Pat. No. 6,383,748 discloses an analytical test device, with a substrate with through going channels. If the sample is provided with a liquid sample of predetermined size, a drop will form, which may be pumped or pressed through the substrate, in order to bind the analyte to a binding material. Under the substrate, the sample will form a droplet, which may be pumped back through the substrate. In this way, the sample droplet may be pumped through the substrate a number of times, to improve mixing and/or binding to the binding material.

A disadvantage of this system is that it may not be used flexibly or efficiently. The sample size must be well controlled and within predetermined limits Moreover, if the drop falls off the substrate, the analysis is lost or at least much less reliable. Furthermore, the substrate size should be adapted, to prevent pumping of gas through the substrate, thus bypassing the liquid sample. Some substrates have a high bubble pressure when wetted, ensuring that liquid may be pumped through relatively easy, while gas may not. Other substrates do not show this difference. The known device does not offer this versatility.

An object of the present invention is to provide a device of the kind mentioned in the preamble of claim 1, that may be used more efficiently and/or flexibly.

This object is achieved by the invention with a device according to claim 1. In particular, because the device further comprises a separate return channel, for connecting the first volume and the second volume and thus allowing flow of the sample fluid from one of the first volume and the second volume to another of the first volume and the second volume, and a return channel valve that is able to close off the return channel, it is now possible to pump a liquid sample of substantially any size through the substrate, and pump it back to the opposite side of the substrate any number of times, through the separate return channel. There is no risk of losing the analysis because a sample droplet falls off the substrate. The device thus allows a more flexible way of analyzing. Furthermore, since the sample may have any size, it allows easier and more efficient analyses.

With the aid of the device and method, to be discussed below, of the present invention, it is easier to develop suitable remedies for the preparation of medicaments for the treatment of the so diagnosed ailment. For instance, the detection in a sample (say, blood) from an organism (say, a human) of a pathogen (say, a bacteria) may thus lead to the diagnosis and the corresponding treatment (say, an antibiotic).

In the description, the embodiments and the claims, the terms “first volume” and “second volume” should be deemed interchangeable, in that they solely serve to discern the two volumes. For example by flipping the device upside down, or by introducing the sample fluid in the second volume, or in any other way, the two terms may be interchanged.

Moreover, the expression “in fluid communication” is intended to mean that the fluid (liquid or gas) is able to contact the surface or volume by simply flowing towards that surface or into that volume (without passing a substrate), as in communicating vessels. It is not intended to be limited to those cases that there is actually a fluid present that contacts the surface or is present in the volume.

Furthermore, it is noted that each of the first and second volume may comprise a number of subvolumes, for example to guide sample fluid to different parallel parts of the substrate with different binding substances, or to more than one substrate. Functionally, and for the purpose of this document, these subvolumes are considered to be one volume, either a first volume or a second volume.

The return channel valve may be an active valve, that is controllable by a valve control device, or e.g. a one-way valve.

The expression “channel” comprises, for the purpose of the present invention, not only straight-walled paths from one end to another, but rather more generally any physical path for fluids between one end of the substrate and another. Such channels may thus also comprise random fluid paths, such as curved or irregular paths, ramifications, a collection of interconnected voids in the substrate, et cetera. The substrate may thus comprise e.g. sponge-like materials having such interconnected voids, but also non-woven fabrics having numerous fluid paths between the fibres, and so on.

It is explicitly noted that the expression “pump” comprises, for the purpose of the present invention, both active and passive pumps. Herein, a passive pump is intended to mean a device that comprises a closed chamber or the like with a variable volume, such as a pump volume with a flexible wall. Here, such a pump is called passive since it does not actively build up a pressure but serves to pass on a pressure exerted thereon by another, external pump. Active pumps are able to establish a pressure difference by themselves. In other words, a “pump” in the present context refers to a closed space with a moveable part for changing the volume of the part. All this will be further elucidated below.

Many substrates used show a bubble pressure in the order of several bars, while the fluid pumping pressure is in the order of several tens of millibars up to several hundred millibars, although of course other values are possible. Such pressures allow pumping the sample fluid that is collected on one side of the substrate through the substrate to the other side thereof. There, the sample fluid will come off the substrate. In other words, the substrate is contacted by gas on the other side. Hence, if now the pressure (difference) would be reversed, only gas would be pumped through the substrate. However, due to the increased pressure required therefor, this will not happen, and the pressure will increase in the second volume. In other words, the substrate acts as a one-way valve, such that the sample fluid may be pumped out of the second volume.

However, in the case of a substrate in which the bubble pressure and the fluid pumping pressure do not differ substantially, this will not function properly, as gas may pass through this substrate relatively easy. Hence, in a special embodiment, at least one of the first volume and the second volume comprises a contact volume directly in contact with the substrate, a reservoir volume, and a valve between the contact volume and the reservoir volume. This embodiment, with a dividable volume, allows the use of a substrate having a relatively low so-called bubble pressure, as compared to the fluid pumping pressure for pumping a sample fluid through the sample. Herein, bubble pressure relates to the pressure difference across the substrate that is required to pump gas through the substrate. To do that, fluid in the substrate must be displaced against the capillary action of the substrate. This bubble pressure may be much higher than the (sample) fluid pumping pressure, which is taken to be the pressure that allows fluid to pass the substrate.

Sample fluid pumped through the substrate towards the volume on the second, or opposite, side of the substrate, will not collect in the volume directly contacting said second or opposite side, i.e. the contact volume, but will flow on, through the valve, herein sometimes referred to as contact volume valve, and will collect in the reservoir volume. The contact volume valve may take over the function of the high bubble pressure of other substrates by closing off the reservoir volume. All other functionalities of other embodiments mentioned herein may then also apply to substrates with not very different pumping pressures and bubble pressures. In other words, the embodiment with the second volume comprising a contact volume, a reservoir volume and a contact volume valve is even more versatile as to the selection of substrate, however at the cost of a higher parts count.

In a special embodiment, the substrate has a bubble pressure in a wetted condition that is higher than a sample fluid pumping pressure of said substrate in a wetted condition. As described above, a substrate which, when wetted e.g. by the sample fluid, has a high bubble pressure, the substrate may function as a gas barrier, while allowing the flow of sample fluid therethrough. Providing the device with such a substrate allows a simpler design, without the need for a contact volume and contact volume valve. By providing a pressure (difference) between the first volume and the second volume that is between the bubble pressure and the sample fluid pumping pressure, the sample fluid will be pumped through the substrate while gas is still trapped by the substrate.

Advantageously, said bubble pressure is at least 10% higher, preferably at least 50% higher, and even more preferably at least 200% higher than said sample fluid pump pressure. When the bubble pressure is at least 10% higher, it is relatively easy to establish a suitable pressure difference, between the sample fluid pumping pressure and the bubble pressure, allowing fluid flow without gas flow. Furthermore, not too critical variations of either or both of the bubble and pumping pressure, e.g. due to binding material to the substrate, do not affect the proper functioning of the device. When the bubble pressure is at least 50% higher, it is not only easy to establish a working pressure difference, but the pressure difference may be selected such that the sample fluid flow rate is in a useful range, since a higher pressure difference ensures a higher flow rate. In particular, when the bubble pressure is at least 200% higher than the sample fluid pumping pressure, a very useful sample fluid flow may be established. Note that other relative differences between bubble pressure and sample fluid pumping pressure may still lead to useful results.

In the above discussion, only relative differences have been discussed. It is alternatively also possible to select the substrate such that the absolute difference between the bubble pressure and the sample fluid pumping pressure is as high as possible, or at least higher than a desired amount. In particular, but not limiting, the bubble pressure is at least 100 mbar, preferably at least 1 bar higher than the sample fluid pumping pressure, for a wetted substrate, with similar advantages as mentioned above. Again, other differences may also lead to desirable results.

In a particular embodiment, the device comprises a wall around at least one of the first volume and the second volume which is at least partially transparent. Said at least partially transparent wall allows detection of DNA etc. on the substrate without removing it from the device. Of course, simple visual inspection may also be allowed by such a transparent part.

Transparent is intended to comprise at least: transparent to visible light, and to ultra-violet and infrared radiation, although transparency for other types of radiation is also contemplated. The at least partially transparent wall may be provided as the wall material itself, as a separate transparent part in a hole in the wall (i.e. a window), etc.

In a special embodiment, the device further comprises a detection system. Providing a detection system makes the device as a whole more versatile, and it is easier to match the analysis device to particular products to be detected. The detection device may itself comprise a transparent window, or be provided in an operative position with respect to a window, a hole in the wall, et cetera.

The detection device may comprise any suitable known detection system, such as an optical detection system, e.g. fluorescence detection. If desired, the analysis device, and/or the detection device, may comprise additional parts, such as a light source, a filter etc., required for its functioning, e.g. detecting the analyte bound to the binding material. These additional parts are only optional in the analysis device.

The device may detect based on label, length, mobility, nucleotide sequence, mass or a combination thereof. In certain embodiments the device can detect based on optical, electrochemical, magnetic principles. In principle any suitable detection device known from prior art may be used.

In certain embodiments, the system also comprises a data collection device to collect data obtained from the detection device.

In certain embodiments, the system also comprises a data processing device to process the data.

In a particular embodiment, the analysis device according to the invention further comprises a sample fluid introduction device. This sample fluid introduction device is not particularly limited. It may for example comprise simply an introduction opening and/or a introduction channel, preferably with a closing valve. After introducing the sample, said valve may be closed, and a completely closed device is provided, or at least possible. Any other embodiment of the sample fluid introduction device is also contemplated, e.g. those allowing (substantially) contamination-free introduction of a sample fluid.

In a special embodiment, the device of the invention is substantially closed. Of course, during introduction of the sample fluid, there is a connection with the outside world. However, it is intended that the analysis device is at least substantially completely closeable, by means of closure means present on or in the device. This may be achieved e.g. by providing valves on all possible channels to the environment. The big advantage is that the device may provide analysis with less risk of contamination, e.g. through exogene DNA from an operator.

In particular, the invention provides a substantially closeable cassette comprising the detection device according to the invention. Such a cassette is preferably compact and portable, such that it may be easily employed for use in situ. It may preferably comprise any other desired device, such as for storage of fluids, one or more pumps, et cetera, such as described in this application, or otherwise known to the skilled person. Advantageously, the cassette is disposable, in order to prevent contamination when reusing such a cassette. It is still possible however to provide a reusable cassette according to this invention.

In a particular embodiment, the return channel flows out into the first and/or second volume opposite the substrate. This allows optimum use of e.g. gravity to collect the sample fluid, and ensures effective pumping of the sample fluid. This effect is improved even further if the wall around the first volume and/or the wall around the second volume has a shape that tapers towards a respective opening in the wall, that connects the respective volume with the return channel. This reduces the risk that sample fluid remains behind in the first or second volume.

In an embodiment, the pump comprises a pump chamber and a moveable part, the pump chamber and the moveable part defining a pump volume that is in fluid communication with the first volume. In principle, it is sufficient when the pump comprises a pump volume, the size of which can be changed, in order to establish a pressure or pressure difference. Thereto, said pump volume may be sealed by a moveable part. “Moveable” should not be limited to “displaceable as a whole” but also to flexible, resilient or the like.

In this embodiment, the pump chamber may be considered to comprise the house of the pump, in which the “moveable” part may move. The pump volume is then defined, or delimited, by the pump chamber and the moveable part. Said moveable part may comprise a piston, a flexible wall, such as a membrane, and so on. The moveable part may be actively moveable, such that displacement of the part is the actual cause of pressure change, while it may also be a passively moveable part, which moves, or displaces etc., because the pressure across it is changed. The latter may e.g. the case when a flexible membrane is used in combination with an external pump.

Note that the terms “first volume” and “second volume” do not relate to specific functions, but merely as ordinal numbers to discern the two. The names may be interchanged, as may the functions. For example, a pressure build up in the first volume may have the same effect on the pressure difference between the first and second volume as a pressure decrease in the second volume, or a suitable simultaneous pressure change in both volumes.

In a special embodiment, the pump volume is also in fluid communication with the second volume, wherein a first pump valve is provided between the first volume and the pump volume, and a second pump valve is provided between the second volume and the pump volume. This embodiment provides complete control over the pressure in both volumes, with the least number of pumps. The pressure in both volumes may be increased or decreased independently, with in principle only a single pump.

It is of course possible to provide more than one pump, or even more than one pump per volume, e.g. parallel pumps, in order to increase the capacity. A special embodiment of the device further comprises an additional pump, that is operatively connected to the second volume. Such embodiment also allows full control over the pressure in both volumes. However, in addition, this embodiment may still function when one of the two pumps is malfunctioning. It may also be advantageous to operatively connect both the pump and the additional pump to both the first and the second volume.

In a particular embodiment, the additional pump comprises an additional pump chamber and an additional moveable part, the additional pump chamber and the additional moveable part defining an additional pump volume that is in fluid communication with the second volume. As mentioned above for the pump, the additional moveable part may be displaceable as a whole, flexible etc., and may comprise a piston, a flexible membrane etc. In all such pumps, use of a flexible membrane has an advantage that the pump or additional pump may be made gas-tight, which is much more difficult when using e.g. a piston or other displaceable part.

In a special embodiment, the moveable part of the pump and the additional moveable part of the additional pump comprise a substantially continuous flexible membrane. This embodiment comprises both the case that both pumps each comprise a continuous membrane, but also the case that the membranes of both pumps together form one continuous membrane. This latter embodiment is even more advantageous in that it is even easier to ensure a gas-tight design of the device, by providing a single gas-tight membrane, without the risk of leakage around the borders of a number of separate membranes.

In a second aspect, the invention provides an analysis method for analyzing a sample fluid for the presence, absence or amount of an analyte in the sample, the analysis method comprising providing an analysis device according to the invention, supplying a sample fluid in said first volume, performing a desired number of times the following steps: operating the pump to establish a pressure difference between the first volume and the second volume such that at least a part of the sample fluid flows from the first volume to the second volume through the substrate, wherein the valve in the return channel is in a closed position, opening the return channel valve and operating the pump to establish a pressure difference between the first volume and the second volume such that at least a part of the sample fluid flows from the second volume to the first volume through the return channel. The substrate is now ready for a detection step. This method allows advantageous use of the device according to the invention, in that sample fluid may be pumped through the substrate any desired number of times. This increases the accuracy of the analysis, both by improving the amount of analyte bound to the binding material, and by improving mixing of the constituents of the sample fluid. Herein, the amount of sample fluid is substantially irrelevant, which makes the method more versatile and robust.

In particular, the method further comprises the step of equalizing the pressure between the first volume and the second volume. In this way, it is prevented that overall pressures keep increasing or that residual pressures interfere with the method. Equalizing the pressure may be performed e.g. after the sample fluid has flowed from the first volume to the second volume, or vice versa, through the substrate or through the return channel, or even only after one or more of all the pumping steps, such as just before actually optically etc. analyzing or inspecting the substrate with the fluid. Equalizing the pressure may be brought about by opening one or more suitable valves, by operating one or more pumps and the like.

In particular, the desired number of times is two or more. Repeatedly performing the sequence of steps improves the sensitivity of the analysis. Any number, such as ten or more, is possible. Note that the desired number of times may be determined dynamically, that is, during performing the method. For example, the desired number of times may be determined depending on the strength of a measurement signal or absence thereof.

In a special embodiment of the method, a detection step is carried out on the substrate still present between the first volume and the second volume. In other words, the substrate is not moved after the pumping actions, in order to prevent possible contamination. Thereto, it is possible to carry out the detection from the side of the substrate where there is little or no sample fluid, in order not to disturb the analysis, such as a fluorescence detection. In the method as described, this may be the second volume side. Alternatively, it is possible to carry out another step of pumping the sample fluid through the substrate, and carry out the analysis from the first volume side of the substrate. If required, the analysis device as provided may comprise a window enabling such optical (or other) detection.

In a special embodiment of the method, the analyte comprises DNA, RNA, polynucleotides, oligonucleotides, polysaccharides or proteins. Detection of such substances may require very accurate analysis in order to establish the presence or absence of e.g. pathogenic organisms or DNA etc. thereof. The present method, with its increased sensitivity through repeatedly pumping the sample fluid through the substrate, provides advantages for such analyses.

In a particular embodiment of the method, the substrate is placed substantially horizontally. This improves the accuracy of the method, in that it is easier to ensure that each part of the substrate receives equal amounts of sample fluid.

In a special embodiment of the method, the first volume is positioned above the substrate with respect to the direction of gravity. This ensures that the sample fluid that is pumped to the second volume is always present in a layer above and in contact with the substrate. This reduces the risk of formation of bubbles which would hinder the pumping through of the sample fluid. Either the bubble pressure of the substrate is high, and thus the pumping action would be hindered mechanically, by a counterpressure from the bubbles. Otherwise, in case the bubble pressure is relatively low and the bubbles would also be pumped through the substrate, the substrate would receive less sample fluid there, which would decrease the sensitivity of the device and method. Furthermore, this configuration reduces the sensitivity for variations in the amount of sample fluid to be processed.

A general remark is that the time required for pumping the sample fluid once through the substrate depends on the applied pressure difference. By controlling said pressure difference, the time may be actively controlled.

The invention may be more clearly understood after reading the description of exemplary embodiments, with reference to the appended drawings, in which:

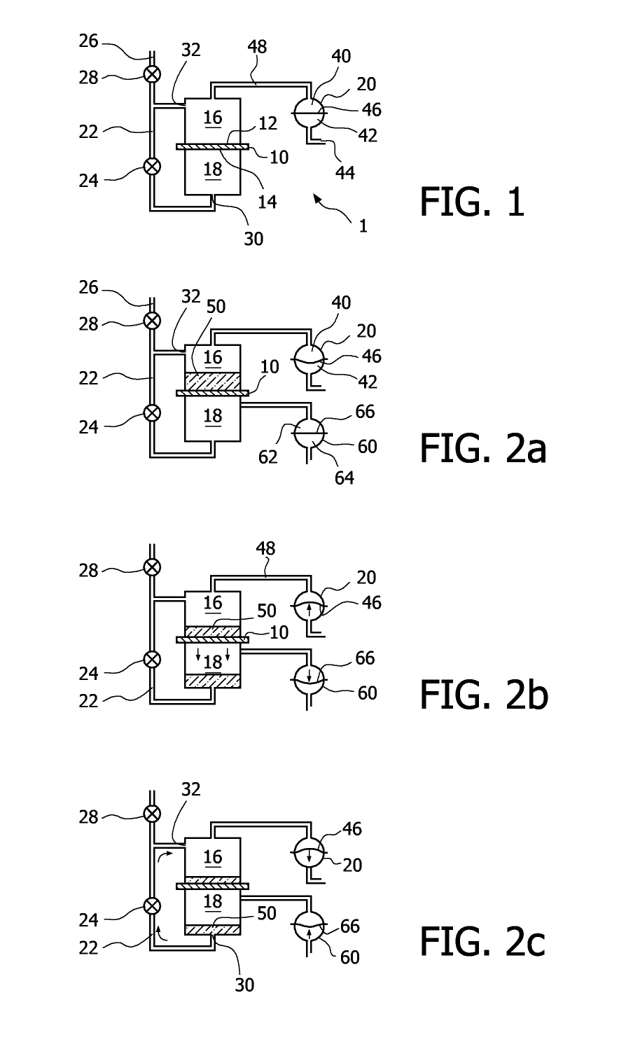

FIG. 1 diagrammatically shows a first embodiment of the device according to the invention;

FIG. 2a-2c diagrammatically show use of an alternative embodiment in the method according to the invention;

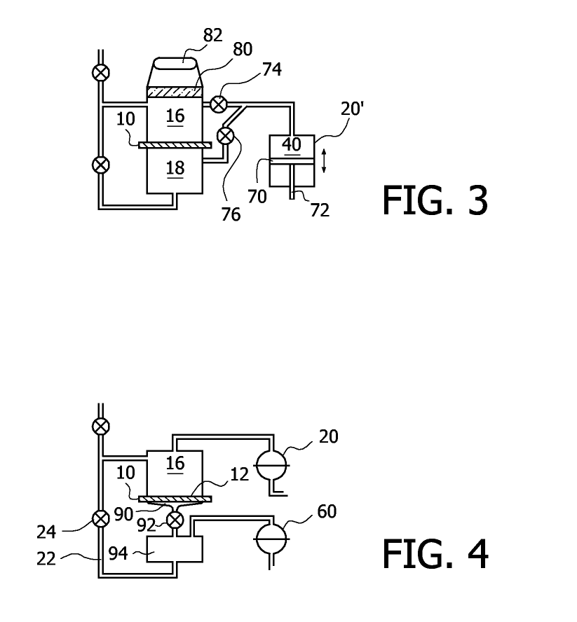

FIG. 3 diagrammatically shows yet another embodiment of the device according to the invention;

FIG. 4 diagrammatically shows yet another embodiment of the device according to the invention; and

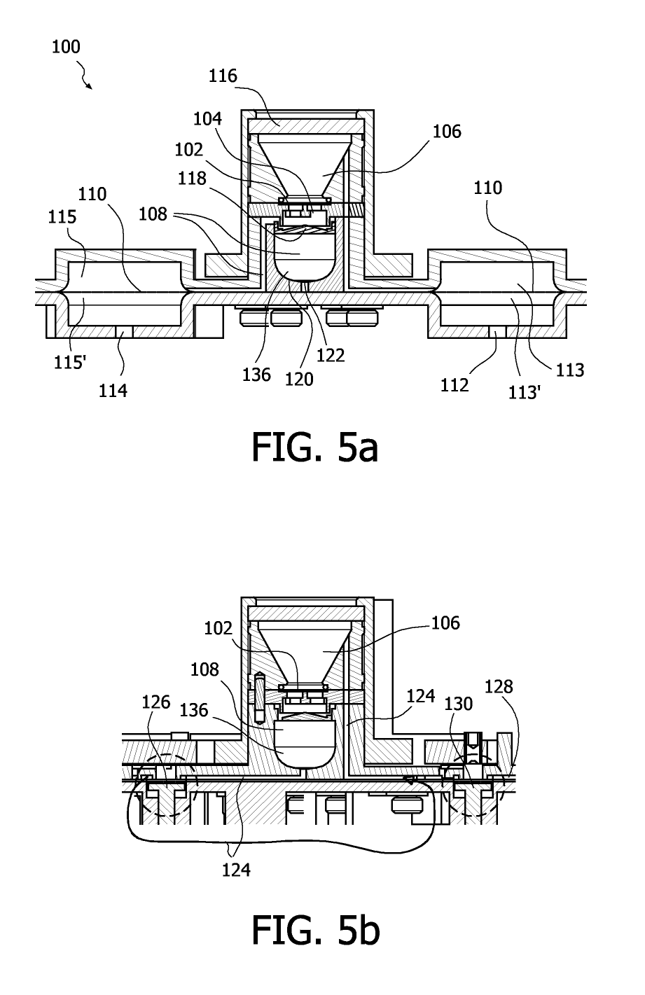

FIGS. 5a and 5b diagrammatically show yet another embodiment of the device according to the invention, and a detail thereof, respectively.

FIG. 1 diagrammatically shows a device 1 according to the invention. Herein, 10 denotes a porous substrate with a first surface 12 and a second surface 14. 16 denotes a first volume and 18 denotes a second volume.

A pump is denoted by reference numeral 20. A return channel 22 connects the first volume 16 and the second volume 18 via first opening 32 and second opening 30, and may be closed off by means of return channel valve 24.

Sample fluid introduction device 26 may be closed off by means of sample valve 28.

Pump 20 comprises a pump volume 40 and a counter volume 42 with a pump inlet opening 44, and is divided by flexible membrane 46.

The porous substrate 10 may be any suitable type of substrate known in the art. For example non-woven fabrics, substrates based on polished and etched hollow fibres of glass or other materials may be used, electroformed substrates, and so on. Preferably, the substrate is at least partly transparent for radiation, preferably optical radiation, such as ultraviolet, visible light or infrared. This improves the detection possibilities for the substrate.

The substrate 10 comprises throughgoing channels, connecting first volume 16 and second volume 18. If the substrate is wetted, it may show a high so called bubble pressure. This means that gases may only pass the substrate when a relatively high pressure is exerted. This bubble pressure may be several bars. Contrarily, liquids may pass relatively easily through the substrate, requiring only modest pumping pressures of e.g. only a few mbars, although of course the pumping pressure may be selected higher, such as e.g. 0.5 bar, in order to increase the flow of fluid through the substrate. All this depends amongst others on capillary pressure in the channels. The device 1 shown in FIG. 1 is particularly suited for substrates 10 with a high bubble pressure. It is also possible to provide a substrate 10 in which the bubble pressure and the pressure required to pass liquid through the substrate do not show such a large difference, but are more or less comparable. A device particularly suited for such substrates is shown in FIG. 4 below. It is noted that FIG. 1, as well as the other Figs. shown, are in principal suited for substrates with a high bubble pressure, while they may be used for substrates having comparable liquid pumping pressures and bubble pressures, if need be with the adaptation as in FIG. 4.

The above discussion of passing liquid and/or gas through the substrate 10 in particular holds for a substantially horizontal positioning of the substrate. When positioned horizontally, the substrate 10 may be wetted evenly, and liquid will pass more or less homogeneously through the substrate 10. This has a positive influence on detection homogeneity and accuracy. Nevertheless, the substrate 10 may be positioned tilted or even vertically, although this may influence said detection homogeneity.

It is noted that the terms “first volume” and “second volume” are interchangeable. This means that these terms and expressions are solely used to discern between two separate volumes 16 and 18. Their functions may be interchanged throughout this application.

The sample fluid introduction device 26 has been indicated only very diagrammatically as a kind of introduction channel. In principle, any desired introduction device known in the state of the art may be provided. The sample fluid introduction device 26 may be closeable by means of sample valve 28. Note that, when the sample valve 28 is closed, the device 1 comprises a completely closed system, comprising the volumes 16, 18 and 40. This greatly reduces the risks of contamination.

Use of the device 1 will be explained in more detail in FIGS. 2a-2c. It is however noted that the pressure difference between first volume 16 and second volume 18 may be established and/or released by means of pump 20 and closing and/or opening of return channel valve 24. For example, in case the sample fluid has been introduced in first volume 16, this may be pumped through the substrate 10 to the second volume 18 by increasing the pressure in first volume 16. Thereto, the pump 20 may move the flexible membrane 46 in the direction of pump volume 40, for example by introducing pressurized gas in counter volume 42 through pump inlet opening 44. Herein, return channel valve 24, as well as sample valve 28 are closed. Under the influence of the increased pressure in first volume 16, the sample will flow through the substrate 10 towards second volume 18.

When the desired amount of fluid has been pumped through substrate 10, the pressure is released. In order to pump sample fluid back from the second volume 18 into the first volume 16, the return channel valve 24 is opened and the pressure in the first volume 16 is lowered, for example by exhausting counter volume 42, which will cause the flexible membrane 46 to move such that the pump volume 40 will increase. Any liquid that has collected at the bottom of the second volume 18, near the second opening 30, will be pumped to the first volume 16 through the return channel 22. This is caused by the pressure in the second volume, which has been increased by the added simple fluid, keeping in mind that gas may not pass through the substrate 10. If this pressure increase is not sufficient to pump the sample fluid back to the first volume 16, the pressure in said first volume may be decreased by reversing the pump action of the pump 20, before opening the return channel valve 24. Subsequently, the return channel valve 24 is closed, and the cycle may be repeated.

It is noted that the pump 20 may comprise a moveable part, here in the form of a flexible, and substantially gas-tight membrane 46, that may actively change the volume of pump volume 40, and hence the pressure in the first volume 16. Alternatively, the pump 20 may be connected to separate pump means (not shown) via pump inlet opening 44. In that case, the flexible membrane, or the moveable part in general, may be a passive part.

FIGS. 2a-2c diagrammatically show an alternative embodiment of the device according to the invention, as well as a use thereof.

Throughout the drawings, similar parts are denoted by the same reference numerals. Hence, only relevant parts will be renumbered.

FIG. 2a shows an alternative embodiment, comprising an additional pump 60, having an additional pump volume 62 and an additional counter volume 64, divided by additional flexible membrane 66. Providing an additional pump 60 offers the advantage that in both the first volume 16 and the second volume 18 the pressure may be increased or decreased, independently of each other. Sample fluid 50 has been introduced in the first volume 16 via sample fluid introduction device 26, sample valve 28, apart of the return channel 22 and first opening 32. Note that it is possible to provide a separate introduction channel, not combined with a return channel 22.

The sample fluid 50 is ready to be pumped through the substrate 10. Sample fluid introduction valve 28 and return channel valve 24 are closed. Note that the flexible membrane 46 may have moved towards counter volume 42, in order to accommodate in the pump volume 40 the volume of gas that was expelled from the first volume 16 when introducing the sample fluid 50.

In FIG. 2b, a second step of the method according to the invention is depicted diagrammatically.

Here, sample valve 28 and return channel valve 24 are closed. A pressure difference between the first volume 16 and the second volume 18 is established by the combined action of pump 20 and additional pump 60, such that sample fluid 50 flows through the substrate 10 towards the second volume 18.

To establish the pressure difference, it is possible to increase the pressure in the first volume 16 by means of the pump 20. Herein, the flexible membrane 46 moves in the direction of the arrow. Alternatively, or additionally it is possible to decrease the pressure in the second volume 18 by means of the additional pump 60. Here, the additional flexible membrane 66 moves in the indicated direction. Of course, it is also possible to increase the pressure in both volumes, but more in the first volume 16 than in the second volume 18, or to decrease the pressure in an analogous way.

If sufficient sample fluid 50, for example all of the sample fluid, has been pumped though the substrate 10 to the second volume 18, the pressure difference is released, for example by opening the return channel valve 24, or by releasing the pressure in the pump 20 and/or the pump 60.

FIG. 2c diagrammatically shows another step of the method according to the invention. In order to pump the sample fluid from the second volume 18 to the first volume 16, the return channel valve 24 is opened while an opposite pressure difference is established. For example, pump 20 exerts a pressure on the first volume 16 which is lower than the pressure in the second volume 18 which is exerted by the additional pump 60. This may for example be established by moving the flexible membranes 46 and 66 in the indicated directions. Thereto, the respective counter volumes of the pump 20 and the additional pump 60 may be pressurized accordingly. Alternatively, the moveable parts of the pumps 20 and 60 may themselves be moved in order to actively establish the pressures.

The sample fluid 50 will flow from the second volume 18 through the return channel 22 and the return channel valve 24 to the first volume 16. If the desired amount of sample fluid, for example all of the sample fluid, has been pumped to the first volume 16, the pressure difference is released and the return channel valve 24 is closed. The analysis device is now ready for repeating the cycle.

By thus passing the sample fluid 50 one or more times through the substrate 10, in particular in substantially equal amounts through every part of the sample 10, it is not only possible to obtain very good detection results, in that the amount of DNA or other analytes bound to the binding materials of the substrate 10 is increased, but also the mixing of the constituents of the sample fluid is increased, which is also beneficial for the accuracy of the detection or analysis.

FIG. 3a diagrammatically shows another embodiment of the device according to the invention. Only relevant parts have been indicated with a reference numeral.

Here, the pump 20′ now comprises a pumping volume 40′, the volume of which may be changed by means of a piston 70, connected to a piston arm 72.

The pumping volume 40′ is in fluid communication with the first volume 16 via first volume valve 74, and in fluid communication with the second volume 18 via second volume valve 76.

A transparent window 80 couples a detection device 82 with the first volume 16.

The embodiment shown in FIG. 3 shows a different pump 20′, comprising a piston 70 and a piston arm 72 that may be moved in the direction of the arrows. The pump however serves the same purpose of controlling pressures, and may replace any pump in any other embodiment. Although mechanical control of the pressure through control of the volume of the pumping volume 40′ is easier, it may be more difficult to establish a gas-tight pumping volume 40′. Of course, the pump 20′ of FIG. 3 may be combined with the pump 20 of for example FIG. 1 in order to obtain a well controlled but gas-tight pump. Furthermore, other types of pumping devices may also be considered, such as piezo-electrical devices, thermal expansion pumps, and so on.

The pump volume 40′ may be placed in fluid communication independently with either of the first volume 16 and/or the second volume 18. Thereto, a first volume valve 74 and a second volume valve 76 are provided. Each may be operated independently. When the first volume valve 74 is opened and the second volume valve 76 is closed, the pressure in the first volume 16 may be changed by operating the pump 20′. Analogously, the pressure in the second volume 18 may be changed in the reversed situation with respect to the valves 74 and 76. This allows performing the steps required for the analysis method according to the invention.

The window 80 is transparent for e.g. optical radiation. This allows analysis of the substrate 10, containing analyte that has been bound to binding material, for example through fluorescence lighting. Other detection methods are also possible, which may require different radiation, and thus a different transparency for the window 80. Also provided is a detection device 82, such as a camera, a CCD or the like. Note that the detection device 82 is optional. In other words, the analysis device according to the invention may also be provided without the detection device 82, but with the window 80. It is thus possible to provide the analysis device as a disposable device, without the need for a detection device 82, which is often very complex and expensive.

FIG. 4 diagrammatically shows another embodiment of the device according to the invention. This embodiment is particularly suited for a substrate 10 for which the bubble pressure is comparable to the fluid pumping pressure.

The second volume now comprises a contact volume 90 and a reservoir volume 94, divided by a contact volume valve 92. The additional pump 60 is in fluid communication with the reservoir volume 94.

If the substrate 10 has a bubble pressure which is relatively low, and comparable to the fluid pumping pressure, the substrate will not work as a gas barrier. This would allow gas to escape through the substrate 10 in case of a pressure build-up in e.g. the second volume of FIG. 3. Note that a pressure build-up in the first volume 16 of FIG. 4, when sample fluid is present on the first surface 12 of the substrate 10, would still be prevented. In the embodiment of FIG. 4, in order to prevent gas flow from the pressurized second volume through the substrate 10 towards the first volume 16, the specific set-up as depicted is provided.

First, the normal step of pressurizing the first volume 16 is carried out, in order to pump sample fluid (not shown) through the substrate 10 to the second volume. When the sample fluid reaches the second volume, i.e. the contact volume 90, it will come off of the substrate 10 dropletwise, and flow through the contact volume valve 92 to the reservoir volume 94, where the sample fluid is collected. Substantially no sample fluid will remain in the contact volume 90, especially if the wall thereof has been made hydrophobic, e.g. by lining with a fluoropolymer or the like. Then, individual droplets will form, which easily flow away, e.g. under the influence of gravity, or any other driving force.

As a next step, the contact volume valve 92 is closed, in order to prevent reflow of gas through the substrate 10 towards the first volume 16. Now, the second volume, i.e. in this case the reservoir volume 94, may be pressurized by the additional pump 60. Then, when the return channel valve 24 is opened, the sample fluid will flow from the reservoir volume 94 through the return channel 22 and the return channel valve 24 towards the first volume 16. Substantially no fluid or gas will directly flow through the substrate 10. Subsequently, after closing the return channel valve 24, the cycle may be repeated.

The embodiment shown in FIG. 4 allows the use of substrates with relatively low bubble pressure. In other words this embodiment allows the use of substrates with any value for the bubble pressure.

Alternatively or additionally, and by making the contact volume 90 so thin that no droplets at all will form, but at the most a thin fluid film, an embodiment is obtained in which the influence of droplets on detection measurements, such as by means of fluorescence detection, is minimized. This is because then in the contact volume only a film is formed, while droplets only form in the reservoir volume 94. Since the film may be made much thinner than individual droplets, and also because the film if present is a more or less continuous and homogeneous layer, any background for such detection measurements is minimized and/or homogenized. Note that this advantage is also present if the contact volume valve 92 is omitted.

FIG. 5a diagrammatically shows another embodiment of the device according to the invention, and FIG. 5b shows a detail thereof.

The analysis device 100 comprises a porous substrate 102, supported by a substrate support 104.

The first volume 106 and the second volume 108 are divided by flexible membrane 110, and are pressurizable via first pressure inlet 112 and second pressure inlet 114, respectively.

116 is an optically transparent window and 118 is a shield plate.

The return channel 124 opens out in a drainage opening 122 of the wall 120 of the second volume 108. The return channel valve is denoted by 126.

A sample fluid introduction device is denoted by 128 and a sample valve is denoted by 130.

A first pump comprises a first pump volume 113 and a first counter volume 113′, divided by the flexible membrane 110, while a second pump comprises a second pump volume 115 and a second counter volume 115′, divided by the flexible membrane 110.

The device 100 comprises two pumps, which may be considered passive pumps. The first pump functions by increasing or decreasing the pressure in the first counter volume 113′ by pumping gas into or out of said first counter volume 113′ via opening 112.

The pressure change causes the flexible membrane 110 to bulge into or out of the first volume 108 in the left part of the drawing, thereby increasing or decreasing the pressure in the first volume 108. It is alternatively possible to provide an active pump by providing an actively movable part instead of the flexible membrane 110, but this would increase the risk of gas leakage. Alternatively, it would be possible to drive the flexible membrane 110 directly via an additional movable part. This would constitute active pumps. Alternative pumps are also possible.

The same functioning holds also for the second pump, comprising a second pump volume 115 and a second counter volume 115′, divided by the flexible membrane 110, as well as a second pump in that opening 114. The absence of active moving parts allows the analysis device 100 to be produced more reliably and cheaper. It is easier to provide it as a disposable part, or an exchangeable part, which is connectable to active pumps.

Clearly visible is the optically transparent window 116, which may serve as a viewport for control of the analysis and pumping steps, but also to allow optical access to a detection device and/or radiation required therefor.

The substrate 102 is supported by a substrate support 104, in order to prevent sagging of the substrate 102, due to its own weight or that of the sample fluid, or due to pressure differences. This substrate support 104 ensures a correct positioning of the substrate 102 with respect to sample fluid flowing therethrough. Note that a homogeneous flow through the substrate 102 improves the accuracy of the analysis. In order to improve the accuracy even further, an optional shield plate 118 may be provided. The shield plate 118 is intended to block radiation originating from an amount of sample fluid 136. For example, if a fluorescence detector is used, the analysis might be affected by fluorescence originating from the sample fluid. This parasitic fluorescence might pass through a substrate 102, which may be at least partially transparent. By providing a shield plate 118, e.g. in the form of a rounded or oblique plate, such parasitic fluorescence may be effectively shielded. Note that droplets of sample fluid 136 may drop from the substrate 102 onto the shield plate 188, and from there flow into the lower part of the second volume 108.

A method for using the analysis device 100 will be elucidated by describing FIG. 5b.

In FIG. 5b, only the parts that are relevant for describing the method have been enumerated, similar parts still being denoted by the same reference numerals.

In particular, 126 denotes the return channel valve in the return channel 124, a part of which has been indicated schematically by the large irregular arrow. A sample fluid introduction device is shown as 128, with a sample valve 130. The sample fluid introduction device 128 may simply be a connection to an external fluid sample container. It is also possible to provide more sophisticated introduction devices, which are known in the art.

In use, sample fluid 136 is introduced in the device 100 through the sample fluid introduction device 128, the sample valve 130 been opened. The sample fluid will go to the first volume 106, where a layer on the substrate 102 will be formed. Next, the sample valve 130 will be closed, thereby substantially sealing the device 100 from the environment.

Subsequently, the pressure in the first volume 106 may be increased, for example by increasing the pressure in the first pump (not shown). Sample fluid 136 will flow through the substrate 102 into the second volume 108. In this case, the substrate has a relatively high bubble pressure, in the order of several bar, say 3 bar. The fluid pump pressure is much lower, in the order of several 10 of mbars to several hundred millibars, say 30 or 300 mbar.

Subsequently, the return channel valve 126 is opened and the pressure in the second volume 108 is increased, in order to pump back the sample fluid 136 through the return channel 124 into the first volume 106. Since, in the present embodiment, the return channel 124 is partially out of the plane of the paper, this return channel 124 has been indicated diagrammatically by the dark irregular arrow. After pumping back the sample fluid to the first volume 106, the return channel valve 126 is closed, and the cycle may be repeated.

The number of cycles depends on various criteria. For example, if there is excellent binding between the analyte in the sample fluid 136 and the binding material in the substrate 102, it is possible that a single cycle (or a few) suffices for the analysis. In other cases, a higher number of cycles is required, such as 2, 3 or more. The number of cycles is in principle unlimited.

The shape of the first volume 106 in the present embodiment tapers towards the substrate 102, in order to guide the droplets of sample fluid introduced into the first volume 106 towards the substrate. This shape is advantageous but not necessary. Said guiding affect may be further improved by providing the surface of the first volume 106 with a hydrophobic material or lining material. Droplets hardly adhere to such material and will easily flow towards substrate 102. It is advantageous to prevent sample fluid 136 from adhering to the walls around the first volume 106, because for example parasitic fluorescence of said droplets may disturb the analysis of the substrate 102.

The embodiment shown in the drawings and described above are intended to be exemplary and non-limiting. The scope of the invention is defined by the appended claims, in view of the description above. Similarly, the reference numerals used in the claims solely serve to clarify the claims in view of some embodiments shown. In particular, they do not limit the claims or the parts thereof provided with such reference numerals to the specific embodiments or parts thereof as depicted in the figures. This holds especially for the first and second volumes.

1. An analysis device (1; 100) for analyzing a sample fluid (50; 136) for the presence, absence or amount of an analyte in the sample fluid (50), the analysis device comprising
a substrate (10; 102) having a first surface (12) and an opposite second surface (14), and having a plurality of through going channels from the first surface (12) to the second surface (14), and at least partially being provided with a binding substance specific for the analyte,
a first volume (16; 106; 18; 108) in fluid communication with the first surface (12),
a second volume (18; 108; 16; 106) in fluid communication with the second surface (14), and
a pump (20, 60; 20′) for establishing a pressure difference between the first volume (16; 106; 18; 108) and the second volume (18; 108; 16; 106), the pump (20, 60; 20′) being operatively connected to the first volume (16; 106; 18; 108), characterized in that the device further comprises
a separate return channel (22; 124), for connecting the first volume (16; 106; 18; 108) and the second volume (18; 108; 16; 106) and thus allowing flow of the sample fluid (50; 136) from one of the first volume (16; 106; 18; 108) and the second volume (18; 108; 16; 106) to another of the first volume (16; 106; 18; 108) and the second volume (18; 108; 16; 106), and
a return channel valve (24; 126) that is able to close off the return channel (22; 124).

2. The device of claim 1, wherein at least one of the first volume (16; 106; 18; 108) and the second volume (18; 108; 16; 106) comprises a contact volume (90) directly in contact with the substrate (10; 102), a reservoir volume (94), and a valve (92) between the contact volume (90) and the reservoir volume (94).

3. The device of claim 1, wherein the substrate having a bubble pressure in a wetted condition, with respect to the sample fluid, that is higher than a sample fluid pumping pressure of said substrate in a wetted condition.

4. The device of claim 3, wherein said bubble pressure is at least 10% higher, preferably at least 50% higher, and even more preferably at least 200% higher than said sample fluid pump pressure.

5. The device of claim 1, comprising a wall around at least one of the first volume (16; 106; 18; 108) and the second volume (18; 108; 16; 106) which is at least partially transparent (80; 116).

6. The device of claim 1, further comprising a detection system (82).

7. The device of claim 1, further comprising a sample fluid introduction device (26; 128).

8. The device of any claim 1, that is substantially closeable.

9. The device of claim 1, wherein the return channel (22; 124) flows out into the first (16; 106; 18; 108) and/or second volume (18; 108; 16; 106) opposite the substrate (10; 102).

10. The device of claim 1, wherein the pump (20, 60; 20′) comprises a pump chamber and a moveable part (46, 66; 70, 72; 110), the pump chamber and the moveable part (46, 66; 70, 72; 110) defining a pump volume (40, 64; 40; 113, 115) that is in fluid communication with the first volume (16; 106; 18; 108).

11. The device of claim 10, wherein the pump volume (40′) is also in fluid communication with the second volume (18), wherein a first pump valve (74) is provided between the first volume (16) and the pump volume, and a second pump valve (76) is provided between the second volume (18) and the pump volume.

12. The device of claim 1, further comprising an additional pump (60), that is operatively connected to the second volume (18; 108; 16; 106).

13. The device of claim 12, wherein the additional pump (60) comprises an additional pump chamber and an additional moveable part (66; 110), the additional pump chamber and the additional moveable part defining an additional pump volume (64; 115) that is in fluid communication with the second volume (18; 108; 16; 106).

14. The device of claim 13, wherein the moveable part of the pump and the additional moveable part of the additional pump comprise a substantially continuous flexible membrane (110).

15. An analysis method for analyzing a sample fluid (50; 136) for the presence, absence or amount of an analyte in the sample fluid (50; 136), the analysis method comprising
providing an analysis device (1; 100) according to claim 1,
supplying a sample fluid (50; 136) in said first volume (16; 106; 18; 108),
performing a desired number of times the following steps:
operating the pump (20, 60; 20′) to establish a pressure difference between the first volume (16; 106; 18; 108) and the second volume (18; 108; 16; 106) such that at least a part of the sample fluid (50; 136) flows from the first volume (16; 106; 18; 108) to the second volume (18; 108; 16; 106) through the substrate (10; 102),
wherein the return channel valve (24; 126) is in a closed position,
opening the return channel valve (24; 126) and operating the pump (20, 60; 20′) to establish a pressure difference between the first volume (16; 106; 18; 108) and the second volume (18; 108; 16; 106) such that at least a part of the sample fluid (50; 136) flows from the second volume (18; 108; 16; 106) to the first volume (16; 106; 18; 108) through the return channel (22; 124).

16. The method of claim 15, further comprising the step of equalizing the pressure between the first volume (16; 106; 18; 108) and the second volume (18; 108; 16; 106).

17. The method of claim 15, wherein the desired number of times is two or more.

18. The method of claim 15, wherein a detection step is carried out on the substrate (10; 102) still present between the first volume (16; 106; 18; 108) and the second volume (18; 108; 16; 106).

19. The method of claim 15, wherein the analyte comprises DNA, RNA, polynucleotides, oligonucleotides, polysaccharides or proteins.

20. The method of claim 15, wherein the substrate (10; 102) is placed substantially horizontally.

21. The method of claim 15, wherein the first volume (16; 106; 18; 108) is positioned above the substrate (10; 102) with respect to the direction of gravity.
